(19) United States
(12) Patent Application Publication
(10) Pub. No.: US 2008/0057009 A1
(43) Pub. Date: Mar. 6, 2008

(54) Talc-based sand removing sun care powder.

(76) Inventor: Michael Brett Grunhaus, Nesconset, NY (US)

Correspondence Address: Michael Brett Grunhaus, 52 Forest Ave, Nesconset, NY, 11767, US

(21) Appl. No.: 11/468,498

(22) Filed: Aug. 30, 2006

(51) Int. Cl. A61K 8/97; A61K 8/27; A61K 36/886

(52) U.S. Cl. 424/59; 424/74; 424/641; 424/744

(57) ABSTRACT

A talc-based, scented, colored, deodorizing, moisture absorbing, soothing, antioxidant sun care powder composition used to remove sand particles from one's skin. The method of using said composition involves shaking the powder either into one's hand or onto the affected area of one's body and wiping away sand, moisture, and odor using one's hand. The user's skin will be left soft, smooth, dry, completely free of sand particles, and will retain a pleasing scent.

### CROSS-REFERENCE TO RELATED APPLICATIONS

| | | |
|---|---|---|
| 4,568,539 | February, 1986 | Ashton, et al. |
| 4,913,896 | April, 1990 | Harvey |
| 5,824,294 | October, 1998 | Abramson, et al. |
| 60/712,194 | August, 2005 | Grunhaus, et al. (Provisional) |

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM

Not Applicable

### BACKGROUND OF THE INVENTION

The present invention relates to body powder compositions and, more particularly, to a talc-based sun care body powder composition that will remove sand particles, undesirable odor, and moisture from one's skin, while simultaneously providing skin soothing and antioxidant benefits.

Body powders have long been available to the general public, primarily for absorbing moisture and oils from the body. Body powders have also been used extensively on babies to help prevent diaper rash and to otherwise help maintain dryness. However, none of these prior body powders with talc powder as their main ingredient have been utilized for the specific purpose of removing sand, odor, and moisture from the skin, while providing antioxidant benefits.

For as long as people have been going to the beach, one of their most irritating dilemmas has been how to remove the sand, odor, and moisture off of their body when they are prepared to leave.

Sand particles have a propensity to stick to the body, especially when they are damp, such as in a beach environment. Combined with personal use of oils, lotions, and sunscreens at the beach, sand adheres to the body and becomes practically impossible to remove.

When not removed effectively, these sand particles make their way into bathing wear, socks, shoes, sandals, clothes, the car, and finally, the home. They can cause skin irritation, extreme discomfort, and leave a messy trail in their wake.

People have always struggled with this problem, trying in vain to wipe sand off with their bare hands, towels, etc. Such action never truly removes all of the sand from the skin. Another problem arises when water (from the ocean or public beach faucets) is applied to remove sand particles from the affected area of the body. This temporary solution only induces more sand particles to stick to the now moisture-laden skin.

The odor one acquires after a day at the beach is at best unpleasant. People will often attempt to shower at the beach if there are such facilities—to attempt to get both the sand and smell off of themselves before getting dressed to leave. They do so with extreme hesitance as such facilities are usually unsanitary. As a result, the majority of beachgoers leave the beach very unhappy with their newfound scents, which will now permeate their apparel and mode of transportation.

### BRIEF SUMMARY OF THE INVENTION

It is the object of this invention to provide a talc-based sand-removing sun care powder composition that will completely remove sand particles from the applied to area of the skin.

It is a further object of this invention that moisture will be absorbed from the applied to area of the skin, leaving the skin smooth and dry.

It is a further object of this invention that unpleasant odor will be neutralized on the applied to area of the skin.

It is a further object of this invention that a desirable pleasing scent will be left on the applied to area of the skin.

It is a further object of this invention that soothing benefits will be attained on the applied to area of the skin.

It is a further object of this invention that antioxidant benefits will be attained on the applied to area of the skin.

It is a further object of this invention to provide a method for using said composition by shaking the sand-removing sun care body powder composition either into one's hand or onto the affected area of one's skin and wiping it off with one's hand.

Other body powders use cornstarch as their main ingredient, which, although acts as a good drying agent, unfortunately, has the disadvantage of encouraging the growth of fungi. Talc powder, also an excellent drying agent, does not encourage the growth of fungi and has been shown to have odor absorbing qualities, as well as nominal antibacterial effects. For these reasons, talc powder is generally preferred to cornstarch for use in a body powder.

### BRIEF DESCRIPTION OF THE SEVERAL VIEWS OF THE DRAWING

Not Applicable

### DETAILED DESCRIPTION OF THE INVENTION

The primary ingredient in this sand-removing sun care powder composition will consist of approximately 96.65% cosmetic grade unscented talc powder by weight of the total composition.

Zinc oxide will be mixed into the sand-removing sun care powder composition at approximately 2% by weight of the total composition.

A pleasing fragrance will be mixed into the sand-removing sun care powder composition at approximately 1% by weight of the total composition. Said sun care powder composition will be manufactured in different “flavors”; each consisting of a different fragrance oil or extract. The remaining ingredients for the said sand-removing sun care powder composition as described herein will remain the same for each. Fragrance oils extracts that can be used, but are not limited to; include coconut cream, melon cucumber, pomegranate, vanilla, citrus, etc.).

Powdered colorant (consisting of iron oxide, diiron trioxide, and triiron tetraoxide) will be mixed into the sand-removing sun care powder composition at approximately 0.26% by weight of the total composition. The colorants will give the said powder composition a sand color—to identify it as a beach product, make it pleasing to the eye, fun to use at beach/vacation and other such environments, and differentiate it from ordinary white body powders.

Aloe barbadensis leaf juice_will be mixed into the sand-removing sun care powder composition at approximately 0.08% by weight of the total composition.

Tocopheryl Acetate (Vitamin E) will be mixed into the sand-removing sun care powder composition at approximately 0.01% by weight of the total composition.

Other components can be added if desired. Such components can include mica powdered colorants for shimmering effects on one's skin as well as antibacterial agents such as triclosan, triclocarban, or benzalkonium chloride.

The compositions of the present invention can be prepared by well known mixing or commercial blending procedures utilized in the consumer product and cosmetic industry. The unscented talc powder, zinc oxide, powdered colorant, Aloe Barbadensis leaf juice, Tocopheryl Acetate (Vitamin E), and other ingredients are thoroughly blended and the fragrance is then uniformly mixed therein.

The method of using said composition involves shaking the powder either into one's hand or onto the affected area of one's body and wiping away sand, moisture, and odor using one's hand. The user's skin will be left soft, smooth, dry, completely free of sand particles, and will retain a pleasing scent.

We claim:

1. A talc-based, scented, colored, deodorizing, moisture absorbing, soothing, antioxidant sun care powder composition used to remove sand particles from one's skin.

2. The composition of claim 1 consisting of approximately 96.65% unscented cosmetic grade talc powder by weight of the total composition.

3. The composition of claim 1 consisting of approximately 2% zinc oxide by weight of the total composition.

4. The composition of claim 1 consisting of approximately 1% fragrance (parfum) by weight of the total composition.

5. The composition of claim 1 consisting of approximately 0.26% powdered colorant by weight of the total composition.

6. The composition of claim 5 consisting of iron oxide, diiron trioxide, and triiron tetraoxide.

7. The composition of claim 1 consisting of approximately 0.08% of aloe barbadensis leaf juice by weight of the total composition.

8. The composition of claim 1 consisting of approximately 0.01% Tocopheryl Acetate (Vitamin E) by weight of the total composition.

9. A method of removing sand particles, undesirable odor, and moisture from one's skin, while providing skin soothing and antioxidant benefits, by shaking composition of claim 1 either into one's hand or onto the affected area of one's skin and wiping it off with one's hand.
